(19) United States
(12) Patent Application Publication
(10) Pub. No.: US 2024/0115780 A1
(43) Pub. Date: Apr. 11, 2024

(54) Computer System for Specifying Adjustable Settings of a Blood Treatment Apparatus

(71) Applicants: Fresenius Medical Care Deutschland GmbH, Bad Homburg (DE); Fresenius Medical Care GmbH, Bad Homburg (DE)

(72) Inventors: Bernhard Karow, Bad Homburg (DE); Juergen Klewinghaus, Bad Homburg (DE)

(21) Appl. No.: 18/263,791

(22) PCT Filed: Feb. 1, 2022
(86) PCT No.: PCT/EP2022/052278

§ 371 Date: Aug. 1, 2023

(30) Foreign Application Priority Data
Feb. 2, 2021 (DE) 10 2021 102 333.5

(51) Int. Cl. A61M 1/16; A61M 1/36

(52) U.S. Cl. CPC A61M 1/1613; A61M 1/1656; A61M 1/3609; A61M 2205/502

(57) ABSTRACT

A computer system for specifying/establishing adjustable settings of a blood treatment apparatus, including a calculation device and a display device. The display device is programmed to display a pre-set value of a target treatment parameter for a treatment of a patient using the blood treatment apparatus and a first input interface with optional switches for changing the pre-set value of the target treatment parameter by a user and/or with a second input interface for entering a treatment specification by the user for a treatment of a patient using the blood treatment apparatus. The computer system further includes a first output interface for outputting technical parameter values of the blood treatment apparatus. The calculation device is programmed to determine technical parameter values for the blood treatment apparatus and to output the determined technical parameter values for the operation of the blood treatment apparatus via the first output interface.

### CROSS-REFERENCE TO RELATED APPLICATIONS

The present application is the national stage entry of International Application No. PCT/EP2022/052278, filed on Feb. 1, 2022, and claims priority to Application No. DE 102021102333.5, filed in the Federal Republic of Germany on Feb. 2, 2021, the disclosures of which are expressly incorporated herein in their entirety by reference thereto.

### TECHNICAL FIELD

The present disclosure relates to a computer system, to a blood treatment apparatus, to a system, and to a method. The present disclosure further relates to a digital storage medium, to a computer program product, and to a computer program.

### BACKGROUND

For the purpose of blood treatment, adjustable settings for variable treatment parameters are to be entered by a user, e.g., the clinic staff, at the blood treatment apparatus used for this purpose. The user can initiate the change of such adjustable settings via manual input, for example via input interfaces, e.g., on a screen (touch screen) or on other interfaces which are suitable for input.

Achieving predetermined treatment goals or targets, such as renal dose in dialysis treatment, may require the user to determine or change a number of adjustable settings, some of which interact with each other, so setting the blood treatment apparatus can be a demanding and also error-prone activity.

### SUMMARY

The present disclosure relates to a computer system for assisting the user in setting adjustment values of a blood treatment apparatus, moreover a blood treatment apparatus including a computer system, and a system. The present disclosure is further related to a digital storage medium, a computer program product, and a computer program.

According to the present disclosure, a computer system for determining adjustable settings of a blood treatment apparatus is proposed.

The computer system may be separate from the blood treatment apparatus, may be connected to the blood treatment apparatus, or may be in signal communication with the blood treatment apparatus.

The computer system includes a calculation device and a display device. The latter may be in the form of a GUI (Graphical User Interface) and/or may also be referred to herein as a user interface.

The display device is programmed to display a pre-set target treatment parameter. The pre-set value for the target treatment parameter may be, for example, the desired target value of an ongoing or planned treatment of a patient using the blood treatment apparatus and/or a parameter of the treatment, for example, such as the renal dose.

In addition, the display device or the computer system includes a first input interface via which optionally the, e.g., pre-set value of the target treatment parameter can be changed by a user, e.g., by manual manipulation via the input interface of the display device or optionally a switch, button, etc.

Additionally or alternatively to being programmed to display a pre-set target treatment parameter and to including the first input interface, the display device or computer system may include a second input interface for input by the user of a treatment specification for a treatment of a patient using the blood treatment apparatus. The treatment specification may, purely by way of example, relate to the question of a desired correction of the acid-base balance of the patient being treated or to be treated.

The computer system further includes a first output interface. The output interface is used to output at least one technical parameter value of the blood treatment apparatus.

The calculation device is programmed to determine or assign at least one technical parameter value for the blood treatment apparatus based on values for the target treatment parameter which have been changed via the first input interface and/or based on the treatment specification which has been entered via the second input interface.

Furthermore, the calculation device is programmed to output via the first output interface the technical parameter values determined or assigned for the operation of the blood treatment apparatus.

The calculation device is connected to the display device and/or to the input interfaces preferably in or prepared for signal communication.

The blood treatment apparatus described herein is preferably designed as a dialysis apparatus, which includes a computer system as described herein.

The system described herein includes or consists of one or more blood treatment apparatuses and a computer system as described herein.

Thereby, the blood treatment apparatus is preferably designed as a dialysis device.

In some embodiments, the one or more blood treatment apparatus(es) and the computer system are separate from each other. The term “separate from each other” may herein include, for example, a spacial, physical separation and/or a separation in that there is no signal communication between the blood treatment apparatus and the computer system. In other embodiments, the treatment apparatus(es) and the computer system are in signal communication with each other.

A method described herein, preferably for preparing an upcoming treatment or treatment session of a patient, alternatively during the treatment session to be performed using a medical treatment apparatus. The method includes:
providing a computer system as described herein, a blood treatment apparatus as described herein, or a system as described herein or using such a component already provided;
changing the pre-set value of the target treatment parameter by actuating, by the user, the first input interface, i.e., a switch of the first input device, a button of the first input device, etc.; and/or inputting a treatment specification by actuating the second input interface;
displaying or outputting, optionally via the first output interface of the display device, the adjustable settings for the blood pump and the dialysis liquid pump calculated or determined by the calculation device based on the change in the pre-set value of the target treatment parameter or the inputting of the treatment specification, and/or outputting a treatment-relevant patient parameter, optionally via the second or third output interface;
optionally: confirming or rejecting the displayed change by the user via buttons of the display device;
optionally: transmitting the calculated or determined adjustable setting for the blood pump and the calculated or determined adjustable setting for the dialysis liquid pump of the confirmed change to the control device or closed-loop control device of the blood treatment apparatus; and
optionally, preferably automatically: controlling or regulating the blood treatment apparatus via the control device or closed-loop control device using the calculated or determined values, e.g., using the adjustable settings for the blood pump (101) and for the dialysis liquid pump.

A digital, particularly non-volatile storage medium, as described herein, particularly in the form of a machine readable carrier, particularly in the form of a diskette, CD, DVD EPROM, FRAM (Ferroelectric RAM) or SSD (Solid-State-Drive), particularly with electronically or optically readable control signals, is designed in such a way as to interact with a programmable computer system, so that a conventional computer system is programmed to be a computer system according to the present invention, e.g., when a memory content of the digital storage medium runs on the programmable computer system.

A computer program product, as described herein, includes a volatile or transient program code or one stored on a machine readable carrier or a signal wave, configured to interact in such a way with a programmable computer configuration of a computer system so that the computer system can be reprogrammed to be a computer system as described herein.

Computer program product, for example, can be understood according to the present disclosure as a computer program stored on a medium, an embedded system being a comprehensive system with a computer program (e.g., an electronic device with a computer program), a network of computer implemented computer programs (e.g., client/server-system, a cloud computing system etc.), or a computer on which a computer program is loaded, runs, is stored, is being performed or developed.

The term “machine readable carrier” as is as used herein, refers in certain embodiments of the present disclosure to a carrier, which contains data or information interpretable by software and/or hardware. The carrier may be a data carrier, such as a diskette, a CD, DVD, a USB stick, a flashcard, an SD card or the like, as well as any other storage referred to herein or any other storage medium referred to herein.

According to the present disclosure, a computer program product may be understood as a programmed application (in short: app), for example, particularly for a smartphone, a tablet or another mobile hand-held device.

A computer program as described herein includes a program code, to initiate that a conventional computer system can be reprogrammed to be a computer system as described herein if the computer program is running on a corresponding computer system. According to the present disclosure, a computer program can, for example be taken to mean a physical, distributable software-product, which includes a program.

In all of the afore-mentioned and following statements, the use of the expression “may be” or “may have” and so on, is to be understood synonymously with “preferably is” or “preferably has,” and so on respectively, and is intended to illustrate embodiments according to the present disclosure.

Embodiments according to the present disclosure may include one or several of the features mentioned above and in the following. In this, the features mentioned herein may, in any combination, be subject-matter of embodiments according to the present disclosure, unless the person skilled in the art recognizes a specific combination as technically impossible. Whenever numerical words are mentioned herein, the person skilled in the art shall recognize or understand them as indications of numerical lower limits. Unless it leads the person skilled in the art to an evident contradiction, the person skilled in the art shall comprehend for example the specification of “one” as encompassing “at least one”. This understanding is also equally encompassed by the present disclosure as the interpretation that a numerical word, for example, “one” may alternatively mean “exactly one”, wherever this is evidently technically possible for the person skilled in the art. Both understandings are encompassed by the present disclosure and apply to all numerical words used herein.

Whenever an applicability or a method is mentioned herein, the present disclosure also preferably encompasses a corresponding programming or configuring of a, in particular according to the present disclosure, suitable apparatus or a section thereof.

When it is disclosed herein that the subject-matter according to the present disclosure includes one or several features in a certain embodiment, it is also respectively disclosed herein that the subject-matter according to the present disclosure does, in other embodiments, likewise according to the present disclosure, explicitly not include this or these features, for example, in the sense of a disclaimer. Therefore, for every embodiment mentioned herein it applies that the converse embodiment, e.g. formulated as negation, is also disclosed.

Whenever an embodiment is mentioned herein, it is then an exemplary embodiment according to the present disclosure.

The statements “top” and “bottom” are to be understood here in case of doubt by the person skilled in the art as absolute or relative space indications, which relate to the orientation of the relevant component during its intended use.

Whenever reference is made herein to a pre-set value, or default adjustable setting, this may be understood to be a last adjusted value, a suggested value, a displayed value or a default setting. A factory set value or a value set by the service technician is herein optional but not mandatory.

The first and/or the second input interface can each be or encompass a correspondingly designed touch screen, a rotary switch, a slider, a keyboard or the like.

The first input interface and the second input interface can be identical, i.e., realised by only one, common input interface. Alternatively, they can be different and/or independent of each other.

Taking into account the change made to the pre-set value of the target treatment parameter readable on the display device or the input of the treatment specification when determining or calculating the adjustable settings for components of the blood treatment apparatus, e.g., for blood pump and/or dialysis liquid pump, can be achieved, e.g., based on known algorithms, which in turn can include further parameter values, e.g., from auxiliary tables, etc. These algorithms and/or further parameter values (as well as further adjustable settings belonging to the latter) can, for example, be stored in a storage device of the computer system.

In several embodiments, the changed value of the target treatment parameter or the entered treatment specification can be actively confirmed or rejected by the user. Suitable input options for this, for example a Save or Cancel button, can be provided.

Confirmation may be a simple pressing of an “OK” button or the like.

In some embodiments, confirmation does not involve typing, entering, selecting, etc., of adjustable settings from a plurality of options or even performing a mental calculation.

The target treatment parameter and/or the treatment specification preferably refer to the specific patient being treated at that moment with the blood treatment apparatus or to a specific patient whose blood treatment session with the blood treatment apparatus is due in the near future.

A renal dose may be defined as the quotient of effluent flow or filtrate flow on the one hand and patient weight on the other.

When determining the effluent flow, different flows (e.g., dialysate flow, dialysis liquid flow, substitute fluid flow, calcium flow, citrate flow, net ultrafiltration) may have to be taken into account, depending on the type of therapy. The renal dose can be, for example, the renal target dose (TRD) or the effective renal dose (RRD).

The renal target dose (TRD) can be calculated, for example, using the following formula:

$$TRD = \frac{PostDF + DF + NFRF + \frac{PreDF}{1 + \frac{PreDF}{BF}}}{m}$$

Herein applies:
BF blood flow or blood flow rate
DF dialysis liquid flow or dialysis liquid flow rate
NFRF net fluid withdrawal flow or net fluid withdrawal flow rate
m patient weight
PostDF post-dilution flow or post-dilution flow rate
PreDF pre-dilution flow or pre-dilution flow rate

The effective renal dose (RRD) can be calculated as a function of the duration of therapy t using the formula

$$RRD = \frac{\frac{PostDV}{t} + \frac{DV}{t} + \frac{NFRV}{t} + \frac{\frac{PreDV}{t}}{1 + \frac{\frac{PreDV}{t}}{\frac{BV}{t}}}}{m}$$

whereby applies:
BV processed blood volume
DV dialysis liquid volume
NFRV net fluid withdrawal volume
m patient weight
PostDV post-dilution volume
PreDV pre-dilution volume

In some embodiments of the computer system described herein, the first input interface is programmed when actuated to decrease or increase a target value for the renal dose as a target treatment parameter. In these embodiments, the display device displays a pre-set value for the renal dose, preferably also the change made by the user.

In several embodiments, the second input interface is programmed to initiate or transmit an acid/base correction as an input treatment specification when actuated.

The display device may preferably be able to output the effects of the changes both numerically, for example in the first output interface, in particular as an indication of the technical flow rates of the involved pumps of the blood treatment apparatus and/or in the second output interface as the expected bicarbonate concentration (at the end of the treatment session) in the serum. Additionally, the display device can output the effects of the change highlighted in a graphic, for example in a third output interface.

In certain embodiments, there is also a status display, for example in the form of a bar, which can be used to guide the user as to how the treatment specification, in particular the acid/base deviation, is adjusted with regard to its effect, its measure, its quantity or quality, etc. The graphical representation of the status display preferably serves to enable the user to quickly identify the treatment specification. For example, if a bar is comparatively high up in a window, a frame, a bar display area, it means it is a comparatively marked or strong correction of a metabolic acidosis. If the bar is in the middle, acidosis or alkalosis will be normalised in the long term. If, on the other hand, the bar is displayed comparatively low down, a strong correction of metabolic alkalosis is more likely. A strong correction in this context means, for example, fast or high doses. How strong or fast the correction is at the discretion of the person treating the patient, for example, referring to the patient's weight and/or other determining factors.

In some embodiments, the first output interface is preferably an optical or graphical interface for the user or includes such an interface.

In some embodiments, the first output interface is or includes a common interface with a control device or closed-loop control device of the blood treatment apparatus.

In some embodiments, the computer system described herein or its display device have a second output interface in order to output, in particular to display, via this, treatment-relevant patient data to the user.

In certain embodiments, the second output interface can be designed and/or configured analogously or identically to the first output interface.

In several embodiments, the treatment-relevant patient data or the further parameter values take into account or relate to a desired anticoagulation for or during the treatment, in particular a citrate-calcium anticoagulation.

In some embodiments, the treatment-relevant patient data or the further parameter values take into account or relate to laboratory results and/or other settings of the blood treatment apparatus, in particular treatment modes, etc., with regard to the blood treatment session of the patient to be treated or being treated at that moment.

In several embodiments, the computer system includes a third output interface programmed or configured to display an acid/base correction graph to the user within the third output interface.

In some embodiments, the graph includes or consists of curves of combinations of adjustable settings, in particular for a flow rate of a blood pump and for a flow rate of a dialysis liquid pump, of selectable combinations.

In several embodiments, the computer system is or includes at least one of a mobile device, hand-held device, mobile phone, smartphone, tablet, etc.

In some embodiments, the computer system is a fixed computer terminal, desktop computer, etc., or includes at least one such device. In these embodiments, it may be provided, for example, in a central monitoring room, in particular in an intensive care unit.

In several embodiments, the computer system includes a computer program or application (app for short) that can be loaded onto such a mobile device or other device, or is stored, is executable, will be run or runs on such a device.

In some embodiments, the technical parameter values determined by the calculation device are or encompass an adjustable setting for the blood pump and an adjustable setting for the dialysis liquid pump. In other words, in these embodiments, the calculation device is programmed to display or otherwise output, via the display device, the blood pump adjustable setting and the dialysis liquid pump adjustable setting, which have been determined or calculated based on changing the pre-set value of the target treatment parameter or by inputting a treatment specification, respectively, for acknowledgement by the user as technical parameter values, or as a part thereof, by means of the first output interface.

In several embodiments, the computer system described herein is, or is in signal communication with, a control device or closed-loop control device of the blood treatment apparatus or a part thereof.

Where reference is made herein to a signal connection or communication connection between two components, this may be understood to mean a connection already existing in use. It may also mean that there is preparation for such a signal communication (wired, wireless or otherwise implemented), for example by coupling the two components, such as by pairing, etc.

Pairing is understood to be a process that occurs in the context of computer networks to establish an initial link between computer units for the purpose of communication. The best-known example of this is the establishment of a Bluetooth connection through which different devices (e.g., smartphone, headphones) are connected to each other. Pairing is sometimes also referred to as bonding.

According to the present disclosure, it may be provided in some embodiments to implement the computer system described herein in a control device or closed-loop control device of a blood treatment apparatus, whereby the blood treatment apparatus is or would in turn be configured to a blood treatment apparatus as described herein. In these embodiments, already existing input and output interfaces as well as the calculation device of the blood treatment apparatus could then be advantageously used to implement the computer system as described herein.

In several embodiments, the computer system may be or include a server-based solution in which the user may call, for example via a website, a program running on the server to run or initiate the method as described herein. The computer system may thus optionally include a server. It may include user interfaces and/or user terminals such as a computer, mobile phone or tablet, which are set up to communicate with the server.

In some embodiments, control device or closed-loop control device is programmed to control or regulate the blood treatment apparatus, which includes a blood pump and a dialysis liquid pump, based on the calculated or determined adjustable settings for the blood pump and the dialysis liquid pump of the target treatment parameter changed by the user or the treatment specification entered by the user as technical parameters.

In some embodiments, the calculation device of the computer system is programmed to be able to acquire further (relevant) parameter values for the blood treatment session via existing or further input interfaces. Alternatively, further parameters can be read in, for example from a storage medium provided for this purpose. Alternatively or additionally, in some embodiments, the computer system is programmed to display the further parameter values within existing or further output interfaces. Advantageously, the calculation device may be configured to take these parameter values into account when determining the adjustable settings for the blood treatment apparatus pumps involved.

In several embodiments, the calculation device of the computer system is programmed to display the blood pump adjustable setting and the dialysis liquid pump adjustable setting of the selected combination to the user for acknowledgement using the display device and/or to output them via the output interface, e.g., to a control device or closed-loop control device of a blood treatment apparatus.

In some embodiments, the output interface and the display device may be identical. Thus, an output via the output interface may be a display via the display device, and vice versa.

In several embodiments, the computer system described herein is programmed to output values indicating pre-set values of target treatment parameters, such as a target value for the renal dose and/or the actual flows or the adjustable settings for the pumps mentioned herein, e.g., the blood pump, the dialysis liquid pump, the substitute fluid pump, the calcium pump, the citrate pump, the heparin pump and/or other pumps, for example, via at least one of the aforementioned output interfaces.

The computer system can be programmed to transfer these values, for example, to a logistics system and/or an accounting system. The logistics system and/or billing system, which can be outside the computer system, but can be part of the system described herein, can in turn be programmed to monitor, record, store, issue, invoice, and/or otherwise process delivery flows, orders, consumption and the like. Therefore, the logistics system can be configured to use values transmitted by the computer system to initiate a repeat order of fluids consumed during a blood treatment session, such as citrate solution, dialysis liquid, etc., and a delivery to the clinic in which the blood treatment took place. The billing system may be designed to use the values transmitted by the computer system to bill for services provided by the clinic in connection with the blood treatment session. For example, the changed values of the target treatment parameters or the input of a treatment specification may reflect the consumption of fluids such as heparin, citrate solution, etc. This can be used as the basis for billing the patient or a payer such as an insurance company.

In several embodiments the blood treatment apparatus described herein is designed as a hemodialysis apparatus, hemofiltration apparatus or hemodiafiltration apparatus, in particular as an apparatus for acute, chronic renal replacement therapy or for continuous renal replacement therapy (CRRT).

In several embodiments, the method includes controlling or regulating the blood treatment apparatus, preferably automatically, based on values changed or entered as described herein and technical parameter values determined thereon.

When “programmed” or “configured” are mentioned herein, it is also disclosed that these terms are interchangeable with each other.

Several or all of the embodiments according to the present disclosure, may include one, several, or all of the advantages mentioned above and/or in the following.

An advantage of the present disclosure may be to enable or facilitate the decision of the user, i.e., primarily the attending physician or the attending person, by determining adjustable settings for pumps of the blood treatment apparatus for the upcoming or current blood treatment, which by using them in the treatment (the adjustable settings) may at the same time achieve a changed or adapted target treatment parameter. As the present disclosure makes it possible to rule out unfavorable combinations of the pump settings from the outset, in view of the simplified change or adaptation of the target treatment parameter or the choice of a treatment specification by the user, this can considerably reduce the complexity of the decision-making task for the attending person.

One advantage of the present disclosure can thus further include the fact that changing adjustable settings through the simplification according to the present disclosure can advantageously also give the less experienced user more confidence when making settings.

According to the present disclosure, the probability of errors when finding adjustable settings can be significantly reduced, for example because mental arithmetic steps or the risk of a mistake by the user can be omitted.

Advantageously, the present systems, methods, and devices can also display the changes in the settings in an auxiliary curve which corresponds to a representation via which the users are previously accustomed to having the settings of the flow rates displayed. This can also be helpful for the user who is experienced with the conventional method.

In this way, the patient safety can indirectly also advantageously be increased.

In contrast, the responsibility and responsibility for finding or changing the adjustable settings can advantageously remain with the user. The user thus receives valuable support from the computer system described herein, but without being surprised by its unauthorized action.

If the blood treatment apparatus and the computer system remain separate devices, existing blood treatment apparatuses can be combined with the computer system described herein to form the system described herein. As interventions in the control of the blood treatment apparatus are not required here, there is also no need for a renewed approval of the blood treatment apparatus as a medical device.

### BRIEF DESCRIPTION OF THE DRAWINGS

The present disclosure is exemplarily explained with regard to the accompanying drawing in which same reference numerals refer to the same or similar components. In the figures of the drawing the following applies:

FIG. 1 shows in a highly simplified representation a system with a blood treatment apparatus in addition to a computer system, according to present the present disclosure;

FIG. 2 shows parts of a graphic (or graphic display) as an exemplary part of a display device, e.g., designed as a GUI, as it could be displayed to the user of an exemplary embodiment of the computer system described herein;

FIG. 3 shows an exemplary design of the GUI on a display device of a computer system according to the present disclosure; and

FIG. 4 shows an exemplary course of a method as described herein using an embodiment of the computer system or system according to the present disclosure.

### DETAILED DESCRIPTION

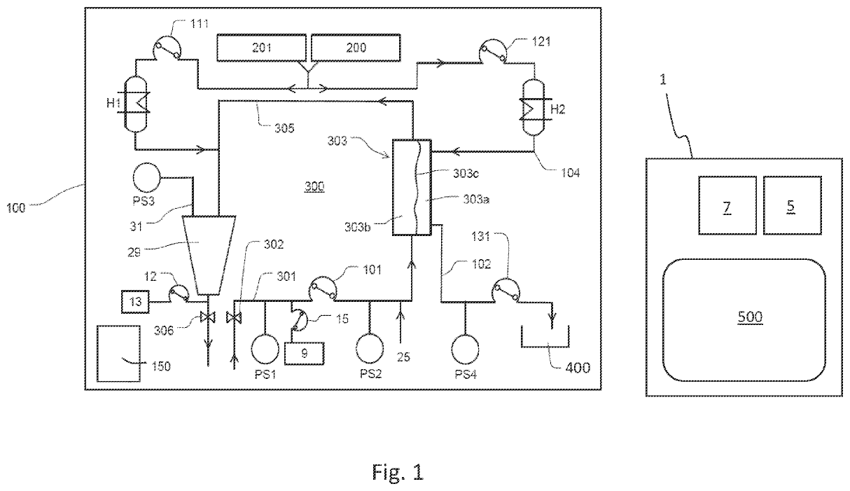

FIG. 1 shows in a highly simplified representation a blood treatment apparatus 100 as part of a system according to the present disclosure, optionally connected to an extracorporeal blood circuit 300.

The extracorporeal blood circuit 300 includes a first line 301, here in the form of an arterial line section.

The first line 301 is in fluid communication with a blood treatment device, here for example a blood filter or a dialyzer 303. The blood filter 303 includes a dialysis liquid chamber 303a and a blood chamber 303b, which are separated from each other by a mostly semi-permeable membrane 303c.

The extracorporeal blood circuit 300 further includes at least one second line 305, here in the form of a venous line section. Both the first line 301 and the second line 305 may be used to connect them to the vascular system of the patient, not shown.

The first line 301 is optionally connected to a (first) tube clamp 302 for blocking or closing the line 301. The second line 305 is optionally connected to a (second) tube clamp 306 for blocking or closing the line 305.

The blood treatment apparatus 100 represented in FIG. 1 schematically and only by some of its devices, includes a blood pump 101. During the patient's treatment the blood pump 101 conveys blood through sections of the extracorporeal blood circuit 300 and in the direction of the blood filter or dialyzer 303 as shown by the small arrows, which generally indicate in each of the figures the direction of flow.

Using a pump for dialysis liquid 121, that may be embodied as a roller pump or as any otherwise occluding pump, fresh dialysis liquid is pumped from a source 200 along the dialysis liquid inlet line 104 into the dialysis liquid chamber 303a. The dialysis liquid leaves the dialysis liquid chamber 303a as dialysate, possibly enriched with filtrate, in the direction of an optional effluent bag 400 and will be referred to herein as effluent.

The source 200 may for example be a bag or a container. The source 200 may further be a fluid line, for example, a hydraulic outlet or hydraulic port of the blood treatment apparatus 100, from which the on-line and/or continuously generated or mixed liquid is provided.

A further source 201 with substitute fluid may optionally be provided. The further source 201 may correspond to the source 200 or be a separate source.

A schematically shown control device or closed-loop control device 150 can be configured to control or regulate the blood treatment session.

Where the optional effluent bag 400 is connected to the blood treatment apparatus 100 is indicated in the bottom right of FIG. 1.

In addition to the aforementioned blood pump 101 and the aforementioned pump 121 for dialysis liquid, the arrangement shown in FIG. 1 further includes, purely optionally, a number of other optional pumps, namely the pump 111 for substitute fluid and the pump 131 for the effluent.

The pump 121 is provided to supply dialysis liquid to the blood filter 303, from a source 200, for example a bag, through an optional available bag heater H2 having a heat bag, using the dialysis liquid inlet line 104.

The thus supplied dialysis liquid exits from the blood filter 303 via a dialysate outlet line 102, (also: effluent inlet line) aided by the optional pump 131, and may be discarded.

An optional arterial sensor PS1 is provided upstream of the blood pump 101. It measures the pressure in the arterial line during a patient's treatment.

A further optional pressure sensor PS2 is provided downstream of the blood pump 101, but upstream of the blood filter 303 and if provided, upstream of an addition point 25 for Heparin. It measures the pressure upstream of the blood filter 303 (“pre-hemofilter”).

To measure the filtrate pressure of the blood filter 303 a further pressure sensor may be provided as PS4 downstream of the blood filter 303, however, preferably upstream of the pump 131 in the dialysate outlet line 102.

Blood, which leaves the blood filter 303, passes through an optional venous blood chamber 29, which may include a de-aeration device 31 and/or a further pressure sensor PS3.

The control device or closed-loop control device 150 shown in FIG. 1 may be in cable or wireless signal connection to any of the components referred to herein—especially or in particular to the blood pump 101—in order to control or regulate the blood treatment apparatus 100.

The optional pump 111 is provided to supply substitute fluid from the optional source 201, for example a bag, via an optional available bag heater H1 having a heat pack, to the second line 305.

From an optionally provided source of citrate solution, here exemplified as a citrate bag 9, in some embodiments a citrate solution is delivered into the line 301, possibly via a citrate pump 15. For example, 4% Na₃ citrate is supplied from the source of citrate solution.

An optional addition device, here designed as a calcium pump 12, is provided to deliver a calcium solution into the line 305 from an optional source of calcium solution, exemplarily designed as a calcium bag 13 in FIG. 1. For example, a CaCl₂-solution is supplied from the source of calcium solution. This can include a calcium concentration of 91 mmol/l, 100 mmol/l or another suitable calcium concentration.

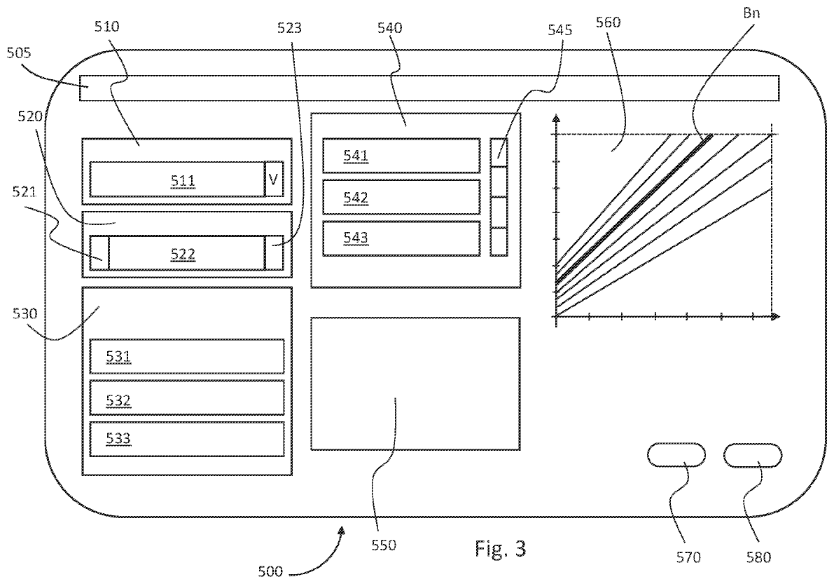

To the right of the blood treatment apparatus 100, FIG. 1 shows a computer system 1 according to the present disclosure including a calculation device 5 and an optional storage device 7. The computer system 1 includes a display device 500. The computer system or display device 500 further includes a first input interface, optionally with switches 521, 523, and/or a second input interface 540 as well as at least one or more output interfaces 530, 550, 560 (not shown in FIG. 1, see FIG. 3).

Blood treatment apparatus 100 and computer system 1 together represent an embodiment of a system according to the present disclosure.

The computer system 1 can be provided on a mobile handheld device, e.g., a mobile phone or tablet, physically separate from the blood treatment apparatus 100. In the embodiment shown in FIG. 1, it is not connected via signal communication to the blood treatment apparatus 100.

The computer system 1 may be or include a server-based solution in which the user can, for example, call up a program running on the server via a website in order to run or initiate some steps of the method described herein. The computer system 1 may thus include a server. The computer system 1 may include user interfaces and/or user terminals such as a computer, mobile phone or tablet, which are arranged to communicate with the server.

However, in embodiments of the blood treatment apparatus other than the one shown here, the computer system is in signal communication and/or physically connected to or part of the blood treatment apparatus 100. In the latter cases, however, the devices connected in this way are no longer a system as described herein, but rather a blood treatment apparatus as described herein.

It is further encompassed by the present disclosure that the computer system 1, other than as shown herein, is identical to or encompassed by the control device or closed-loop control device 150.

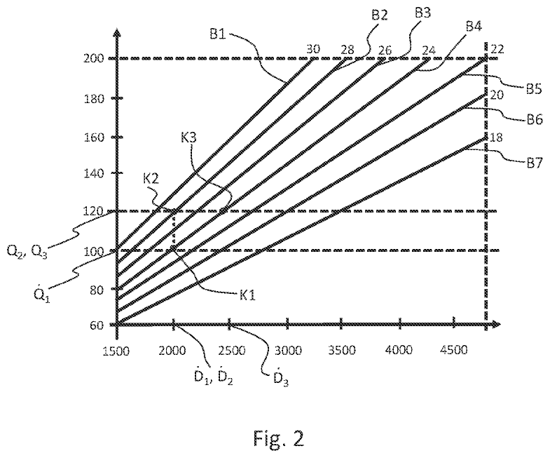

FIG. 2 shows parts of a graphic (or a graphic display) as an exemplary part of a display device 500 (not shown in FIG. 2), e.g., designed as a GUI, as they could be displayed to the user of an exemplary embodiment of the computer system 1 according to the present disclosure.

In the graphical display diagram, the vertical axis represents the blood flow [in ml/min] over the dialysis liquid flow [in ml/h] on the horizontal axis.

Each point within the diagram shown thus corresponds to a combination of a possible adjustable setting \dot{Q}ₙ for the delivery rate of the blood pump on the one hand and a possible adjustable setting \dot{D}ₙ for the delivery rate of the dialysis liquid pump on the other. For example, combination K1 corresponds to a combination of 100 ml/min as the rate for the blood pump (adjustable setting \dot{Q}₁=100 ml/min) with 2000 ml/min as the rate for the dialysis liquid pump (adjustable setting \dot{D}₁=2000 ml/min). Combination K2, on the other hand, stands for 120 ml/min as the rate for the blood pump (adjustable setting \dot{D}₂=120 ml/min) with—as already in combination K1—2000 ml/min as the rate for the dialysis liquid pump (adjustable setting \dot{D}₂=2000 ml/min).

With the possible setting of a series of such combinations K1, K2, K3, . . . from a delivery rate for the blood pump and a delivery rate for the dialysis liquid pump, an expected acid-base-status results mathematically (expected bicarbonate concentration B1, B2, B3, . . . Bn or interim values in the serum).

The expected bicarbonate concentrations Bn in the serum are shown, in the form of rays in the diagram for a number of combinations of blood flow and dialysis liquid flow. The rays shown are indicated as B1 to B7, they are purely exemplary. For each of them, it is indicated at the edge of the diagram which value (in mmol/l) of bicarbonate is to be expected when setting a combination of flows located on each of them (the values in the graph in FIG. 2 were determined in a simulation). With the combinations of blood flow and dialysis liquid flow, adjustable settings can alternatively be selected that would not lie on a straight line in the graphic.

Using the example here, it could be seen, for example, that an increase in blood flow by 20%, from K1 to K2, would change the expected bicarbonate concentration in the serum by around 4 mmol/l upwards (from 24 mmol/l to 28 mmol/l, i.e., towards metabolic alkalosis). An increase in dialysis liquid flow of 20% (to K3) would compensate for this effect.

The underlying constant treatment parameters in this example are: citrate dose 4 mmol per litre of blood, calcium dose 1.7 mmol per litre of blood, net ultrafiltration 100 ml/h.

In several embodiments, when displaying the graph using the third output interface 560 (see FIG. 3), it is intended to highlight an expected serum bicarbonate concentration Bn corresponding to the treatment parameter settings (see FIG. 3).

FIG. 3 shows an exemplary structure of the GUI of a display device 500 of a computer system 1 according to the present disclosure with the possibility of changing pre-set values of the target treatment parameters and/or the possibility of entering a treatment specification, in each case by the user, for the treatment of a patient using the blood treatment apparatus 100 (not shown in FIG. 3).

A field 505 may be provided for displaying general data, for example, the date, time, name of the patient, the patient's weight and/or BMI and/or the like. It is optionally arranged as a heading.

At the top left, below the field 505, an optional window 510 can be seen in FIG. 3, via which, for example, a type of treatment (e.g., treatment mode) can be displayed or indicated at this or another point. Alternatively or additionally, the treatment mode can be changed and/or set via the window 510, or at another location. In this case, the type or mode of the current treatment can be displayed in a field 511 and, for example, via a drop-down menu (see arrow key in field 511 on the right), further types of treatment can be made available and selected or adjusted by the user from the menu.

Below the window 510, there is optionally another window 520 for displaying and/or setting treatment parameters (e.g., treatment settings), in particular the dialysis dose (renal dose) as an example of a target treatment parameter. Here, the pre-set value of the target treatment parameter is displayed in field 522. Using a first input interface, a reduction of the displayed value or an increase of the displayed value of the target treatment parameter, in this case the dialysis dose, can be changed, adapted or set, for example, using the switches 521 or 523, of the first input interface, which may be a button.

In a first output interface 530 which is exemplary designed as a display, important treatment parameters or dialysis parameters (e.g., dialysis parameters), for example the technical flow rates of the pumps 101, 111, 121, 131 involved in the blood treatment, can be displayed as technical parameters. For example, the dialysis liquid flow may be or be displayed in field 531, the blood flow in field 532 and/or the ultrafiltration rate in field 533. These flow rates can be measured values and/or calculated values.

Within an optional second input interface 540, exemplarily shown in the middle of the representation above the display device 500, are found exemplary operating elements 541, 542, 543 for the acid/base correction in the sense of a treatment specification, for example an operating element 541 for the correction of a metabolic acidosis, an operating element 542 for standard settings and/or an operating element 543 for the correction of a metabolic alkalosis. In addition, to the right there is an optional status display 545, which can be used by the user for orientation as to how and to what level (“strong”, “medium”, “weak”, etc.) the treatment specification, in particular the acid/base deviation, is set. The graphical representation of the status display 545 preferably serves for the quick detection of the treatment specification by the user. It can optionally support the information given by means of numerical values. For example, if the bar is high up, a strong or high-dose correction of a metabolic acidosis is desired. If it is in the middle, acidosis or alkalosis will be normalised in the comparatively long term, and if the bar is low down, a strong correction of a metabolic alkalosis is adjusted.

Within the second output interface 550, exemplarily shown in the middle below in the representation of the display device 500, changes can be displayed that would be caused by the correction of the acid/base balance. For example, an expected bicarbonate concentration in the blood towards the end of the treatment session may be or is displayed there.

In the optional graph within the third output interface 560, which can be constructed analogously to the graph in FIG. 2, an auxiliary curve can be highlighted which corresponds to the expected bicarbonate concentration. This is particularly suitable for users who prefer to have the auxiliary curve shown here for determining parameters displayed.

A cancel button 570 can be used to reset selected settings.

Using an optional save button 580, selected adjustable settings can be saved and, possibly, transmitted to the control device or closed-loop control device 150 of the blood treatment apparatus 100.

The technical parameter values determined on the input made above can optionally be transferred from the user interface of the display device 500, which can also be part of the blood treatment apparatus 100, optionally directly—manually or automatically—to the control device or closed-loop control device 150 (not shown in FIG. 3).

In several embodiments, the computer system 1 or at least its input interfaces 521, 523, 540 and/or its output interfaces 530, 550, 560 can be implemented outside the blood treatment apparatus 100, for example on a mobile device, handheld device, mobile phone, smartphone, tablet, etc. or it can be or include an application suitable for this purpose. In this case, the settings must be transmitted to the blood treatment apparatus 100 or its control device or closed-loop control device 150. Suitable components for this can be provided. Transmission by hand by the user is also possible in some embodiments and in some of these it is the only option.

The arrangement of the fields, operating elements and buttons on the display device 500 are purely exemplary and are not to be understood as limiting. Any other arrangement is also encompassed within the present disclosure.

In certain embodiments, further treatment-relevant patient data can be provided, for example a wide variety of laboratory results (e.g., pH, BE, HCO3, pCO2, lactate, etc.), data on anticoagulation and/or a patient history, which also include settings for treatment parameters from previous treatment sessions, can be displayed on the display device 500. This can take place, for example, within the third output interface 560.

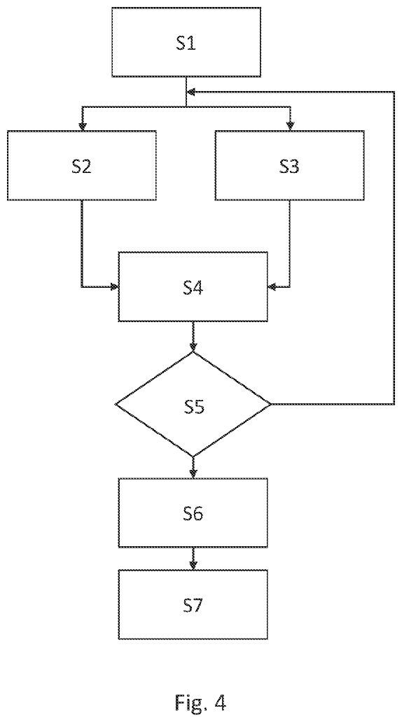

FIG. 4 shows a flowchart of an exemplary embodiment of the method described herein, as it could be initiated or executed via or using the computer system 1 or a system described herein.

Thereby, method step S1 represents providing a computer system 1 according to the present disclosure, a blood treatment apparatus 100, according to the present disclosure or a system according to the present disclosure.

In method step S2, the user by activating the first input interface, e.g., using switches 521 and 523, increases or decreases a preset, previously displayed, last adjusted, etc. target treatment parameter, here as an example the renal dose. Thus, the recommended or the last value taken of the target treatment parameter, for example the renal dose, for the treatment of the patient is changed or adapted using the first input interface on the display device 500 of the blood treatment apparatus 100.

Alternatively or additionally, the input of a treatment specification, for example an acid/base correction, can be initiated via the second input interface 540 in method step S3. For example, a correction of a metabolic acidosis can be selected or initiated by selecting or actuating the operating element 541, for example a correction of a metabolic alkalosis by the selection of the operating element 543.

S4 represents displaying, via the first output interface 530 of the display device 500, the adjustable settings \dot{Q}_{B}, \dot{D}_{B} for the blood pump 101 and the dialysis liquid pump 121 as technical parameter values calculated by the calculation device 5 on the basis of the adjusted renal dose or the acid/base correction initiated, for example as described above. In addition, the output of the expected result of the change, for example the expected value of the bicarbonate concentration, e.g., in the serum towards the end of the treatment, via the second output interface 550, or for example by highlighting the corresponding auxiliary curve, which corresponds to the expected bicarbonate concentration, in a graphic within the third output interface 560, e.g., a diagram.

In method step S5, the displayed change can be confirmed or rejected by the user using buttons 570, 580 on the display device 500. If the changes are discarded, you can optionally return to method step S2 and/or method step S3; if the changes are confirmed, you can continue with method step S6.

In method step S6, the calculated adjustable setting for the blood pump 101 and the calculated adjustable setting for the dialysis liquid pump 121 of the confirmed change are transmitted to the control device or closed-loop control device 150 of the blood treatment apparatus 100. This can be done with a cable or, preferably, wirelessly. It can be done manually.

S7 represents the optional method step of the, preferably automatically, controlling or regulating of the blood treatment apparatus 100 via the control device or closed-loop control device 150 based on the changes made to the target treatment parameter or on the basis of the treatment specification entered, for example by adapting the adjustable setting \dot{Q}_{B} for the blood pump 101 and the adjustable setting \dot{D}_{B} for the dialysis liquid pump 121 to the calculated and transmitted values.

Optionally, the steps described above are carried out in the order mentioned above, overlapping or simultaneously or recursively.

### LIST OF REFERENCE NUMERALS

1 computer system
5 calculation device
7 storage device (optional)
9 citrate solution source, here an example being a citrate bag
12 calcium pump
13 calcium solution source, calcium bag
15 citrate pump
25 addition point for Heparin (optional)
29 venous blood chamber (optional)
31 de-aeration device
100 blood treatment apparatus
101 blood pump
102 dialysate outlet line, effluent inlet line
104 dialysis liquid inlet line
111 pump for substitute fluid
121 pump for dialysis liquid
131 pump for dialysate or effluent in effluent inlet line
150 control device or closed-loop control device
200 source with dialysis liquid
201 source with substitute fluid, optional
300 extracorporeal blood circuit
301 first line (arterial line section)
302 (first) tubing clamp
303 blood filter or dialyzer
303a dialysis liquid chamber
303b blood chamber
303c semi-permeable membrane
305 second line (venous line section)
306 (second) tubing clamp
400 effluent bag
500 display device
505 field for displaying general daily and/or patient data
510 window
511 field for displaying treatment type
520 window
521 first input interface switch (for example, minus)
522 field for displaying target treatment parameters
523 first input interface switch (for example, plus)
530 first output interface for displaying technical flow rates
531 field for displaying the dialysis liquid flow rate
532 field for displaying the blood flow
533 field for displaying the ultrafiltration rate
540 second input interface
541 operating element
542 operating element
543 operating element
545 status display
550 second output interface
560 third input interface
570 cancel-button
580 save-button
K1 combination 1
K2 combination 2
K3 combination 3
B1 expected bicarbonate concentration in serum of 30 mmol/l
B2 expected bicarbonate concentration in serum of 28 mmol/l
B3 expected bicarbonate concentration in serum of 26 mmol/l
B4 expected bicarbonate concentration in serum of 24 mmol/l
B5 expected bicarbonate concentration in serum of 22 mmol/l
B6 expected bicarbonate concentration in serum of 20 mmol/l
B7 expected bicarbonate concentration in serum of 18 mmol/l
Bn bicarbonate concentration
H1 bag heater
H2 bag heater
PS1 arterial sensor
PS2 pressure sensor (optional, “pre-hemofilter”
PS3 pressure sensor
PS4 pressure sensor
S1 method step
S2 method step
S3 method step
S4 method step
S5 method step
S6 method step
S7 method step
Q;\dot{ }₁, Q;\dot{ }₂, Q;\dot{ }₃, Q;\dot{ }_{B} adjustable blood pump settings
\dot{D}₁, \dot{D}₂, \dot{D}₃, \dot{D}_{B} adjustable dialysis liquid pump settings

1-19. (canceled)

20. A computer system for specifying settings of a blood treatment apparatus, the computer system comprising:
a calculation device; and
a display device configured or programmed to display
a pre-set target treatment parameter for a treatment of a patient to be delivered using the blood treatment apparatus, and
a first input interface for changing the pre-set value of the target treatment parameter by a user, and/or a second input interface for entering a treatment specification by the user for the treatment of the patient using the blood treatment apparatus, and
a first output interface for outputting technical parameter values of the blood treatment apparatus,
wherein the calculation device is programmed to:
determine technical parameter values for the blood treatment apparatus based on the changed target treatment parameter value entered via the first input interface and/or the treatment specification entered via the second input interface, and
output the determined technical parameter values for an operation of the blood treatment apparatus, via the first output interface.

21. The computer system according to claim 20, wherein the first input interface comprises switches, and wherein when actuated, the switches of the first input interface are programmed to decrease or increase a target value for a renal dose as a target treatment parameter.

22. The computer system according to claim 20, wherein the second input interface is programmed to initiate an acid/base correction as an input treatment specification when actuated.

23. The computer system according to claim 20, wherein the first output interface is or comprises an optical interface or a graphic interface for the user.

24. The computer system according to claim 20, wherein the first output interface is or comprises an interface with a control device or with a closed-loop control device of the blood treatment apparatus.

25. The computer system according to claim 20, further comprising a second output interface for displaying treatment-relevant patient data to the user within the second output interface via the display device.

26. The computer system according to claim 20, further comprising a third output interface, programmed or configured to display to the user a graphic for acid/base correction within the third output interface.

27. The computer system according to claim 26, wherein the graphic comprises curves with combinations of adjustable settings.

28. The computer system according to claim 27, wherein the adjustable settings comprise selectable combinations of a flow rate for a blood pump and a flow rate for a dialysis liquid pump.

29. The computer system according to claim 20, wherein the computer system comprises a mobile device, a handheld device, a mobile phone, a smartphone, a tablet, a fixed computer terminal, or a desktop computer.

30. The computer system according to claim 29, wherein the computer system is in an intensive care unit and/or a central monitoring room, or comprises an application suitable for an intensive care unit and/or central monitoring purpose.

31. The computer system according to claim 20, wherein the technical parameter values determined by the calculation device comprise an adjustable setting for a blood pump and an adjustable setting for a dialysis liquid pump.

32. The computer system according to claim 20, wherein the computer system comprises or is in signal communication with a control device or a closed-loop control device of the blood treatment apparatus or a part thereof.

33. The computer system according to claim 32, wherein the control device or the closed-loop control device is programmed to control or regulate the blood treatment apparatus, which comprises a blood pump and a dialysis liquid pump, based on the calculated or determined adjustable settings, and based on a target treatment parameter changed by the user or a treatment specification entered by the user.

34. The computer system according to claim 33, wherein the calculated or determined adjustable settings comprise a flow rate for the blood pump and a flow rate for the dialysis liquid pump as technical parameters.

35. A system comprising:
one or more blood treatment apparatuses, each designed as dialysis apparatus; and
a computer system for specifying settings of a blood treatment apparatus, the computer system comprising:
a calculation device; and
a display device configured or programmed to display
a pre-set target treatment parameter for a treatment of a patient to be delivered using the blood treatment apparatus, and
a first input interface for changing the pre-set value of the target treatment parameter by a user, and/or a second input interface for entering a treatment specification by the user for the treatment of the patient using the blood treatment apparatus, and
a first output interface for outputting technical parameter values of the blood treatment apparatus,
wherein the calculation device is programmed to:
determine technical parameter values for the blood treatment apparatus based on the changed target treatment parameter value entered via the first input interface and/or the treatment specification entered via the second input interface, and
output the determined technical parameter values for an operation of the blood treatment apparatus, via the first output interface;
wherein the one or more blood treatment apparatuses and the computer system are separate from each other.

36. The system according to claim 35, wherein the blood treatment apparatus includes a hemodialysis apparatus, a hemofiltration apparatus, a hemodiafiltration apparatus, an apparatus for acute, chronic renal replacement therapy, or an apparatus for continuous renal replacement therapy (CRRT).

37. The system of claim 36, further comprising a digital storage medium having electronically readable control instructions stored therein that when executed on the computer system, causes programming of the calculation device and the display device.

38. A method for preparing a treatment session of a patient, which is to be carried out using a medical treatment apparatus, the method comprising:
receiving a changing of a pre-set value of a target treatment parameter of the treatment session by actuating, by a user, a first input interface of a display device, and/or an inputting of a treatment specification for the treatment session by actuating a second input interface; and
displaying or outputting adjustable settings for a blood pump and a dialysis liquid pump calculated or determined by a calculation device based on a change in the pre-set value of the target treatment parameter or based on the inputting of the treatment specification, and/or outputting a treatment-relevant patient parameter.

39. The method of claim 38, wherein displaying or outputting the adjustable settings comprises displaying or outputting the adjustable settings via the first output interface of the display device.

40. The method of claim 38, wherein outputting the treatment-relevant patient parameter comprises outputting the treatment-relevant patient parameter via a second output interface or a third output interface.

41. The method of claim 38, further comprising receiving an input from the user indicating pressing of buttons on the display device to confirm or reject the displayed changes.

42. The method of claim 38, further comprising transmitting to a control device or closed-loop control device of the blood treatment apparatus the calculated or determined adjustable setting for the blood pump and the calculated or determined adjustable setting for the dialysis liquid pump of a confirmed change.

43. The method of claim 38, further comprising controlling or regulating the blood treatment apparatus via a control device or closed-loop control device using the calculated or determined adjustable setting for the blood pump or for the dialysis liquid pump based on a value change of the target treatment parameter initiated by the user or by the entering of a treatment specification.

44. The method of claim 43, wherein controlling or regulating the blood treatment apparatus is performed automatically.
